(19) United States
(12) Patent Application Publication
(10) Pub. No.: US 2015/0087686 A1
(43) Pub. Date: Mar. 26, 2015

(54) CRYSTALLINE FORMS OF SAXAGLIPTIN

(71) Applicant: ENANTIA, S.L., Barcelona (ES)

(72) Inventors: Nicolas Tesson, L'hospitalet De Llobregat (ES); Montserrat Trilla Castaño, Barcelona (ES); Lydia Cárdenas Romaña, Barcelona (ES); Llorenç Rafecas Jané, Llorenç Del Penedes (ES)

(73) Assignee: ENANTIA, S.L., Barcelona (ES)

(21) Appl. No.: 14/396,370

(22) PCT Filed: Apr. 24, 2013
(86) PCT No.: PCT/EP2013/058504

§ 371 Date: Oct. 22, 2014

(30) Foreign Application Priority Data
Apr. 25, 2012 (EP) 12382158.9
Jun. 15, 2012 (EP) 12382242.1

(51) Int. Cl. C07D 209/52

(52) U.S. Cl. CPC C07D 209/52
USPC 514/412; 548/452

(57) ABSTRACT

It comprises a cocrystal of saxagliptin hydrochloride and a compound selected from the group consisting of saxagliptin, glycolic acid, malonic acid, and urea; or a solvate thereof, or a hydrate thereof. It also comprises a salt of saxagliptin with glycolic acid 1:1 hydrate. It also comprises their preparation processes, as well as their use as antidiabetics.

The present invention relates to crystalline forms of saxagliptin, in particular, to cocrystals of saxagliptin with a second component, and to a salt of saxagliptin, processes for their preparation, and their uses as medicaments. It also relates to pharmaceutical compositions comprising them.

### BACKGROUND ART

Saxagliptin is the International Nonproprietary Name (INN) of (1S,3S,5S)-2-[(2S)-2-amino-2-(3-hydroxy-1-adamantyl)acetyl]-2-azabicyclo[3.1.0]hexane-3-carbonitrile, and has the CAS Nr 361442-04-8. It is a new oral dipeptidyl peptidase-4 (DPP-4) inhibitor developed by Bristol-Myers Squibb.

The structure of saxagliptin in the form of monohydrate corresponds to formula (I):

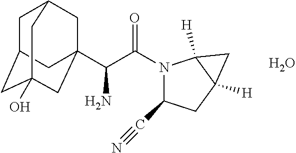

Saxagliptin monohydrate is converted to saxagliptin hydrochloride in situ during drug product manufacturing. Saxagliptin hydrochloride is indicated as an adjunct to diet and exercise to improve glycemic control in adults with type 2 diabetes mellitus.

Different synthetic strategies for the preparation of saxagliptin and its salts are known in the art. For instance, WO2001068603 discloses certain cyclopropyl fused pyrrolidine-based inhibitors of dipeptidyl peptidase 4 including saxagliptin and its salts, method for their preparation and pharmaceutical compositions using these compounds. In particular, Example 60 discloses the preparation of a salt of saxagliptin with trifluoroacetic acid.

WO2008131149 discloses a saxagliptin free base, and hydrates thereof (H.5-2 and H-1), as well as pharmaceutically acceptable salts of saxagliptin other than the trifluoroacetic acid salt. Among these salts, it can be found four hydrate hydrochloride forms of saxagliptin (H2-1, H0.75-3, H1.25-2, H1.67-1), one anhydrate hydrochloride (P-5) and one dihydrated dihydrochloride (H2-1 diHCl) form. However, high-water content forms have certain drawbacks, as a compound prone to degradation by cyclization followed by hydrolysis like saxagliptin can show decreased chemical stability when present in such forms. Moreover, from a galenic perspective, bulk quantities of active pharmaceutical ingredients having high water content tend to clog or stick together, thus sometimes having poor processing behaviour in the formulation processes for the production of pharmaceutical compositions.

WO20100115974 discloses three forms of saxagliptin hydrochloride (I-S, HT-S, IV-S) but with a water content of not more than 1.5% by weight. Unfortunately, the anhydrate HCl forms (P-5, I-S, HT-S and IV-S) seem not very stable under room temperature and transforms to the form of saxagliptin hydrochloride dihydrate H2-1. Inventors have observed also this transformation with hydrates of saxagliptin having a lower amount of water than H2-1, for instance, saxagliptin hydrochloride H0.75-3 transforms to saxagliptin hydrochloride dihydrate H2-1 after one night at room temperature. On the other hand, the saxagliptin hydrochloride dihydrate H2-1 is stable. Unfortunately, it is difficult to prepare it in pure form by an industrial process which could be reproducible at industrial scale.

There are also several patent applications disclosing other specific salts of saxagliptin. WO2012017028 discloses salts of saxagliptin with organic di-acids, in particular, L-malic acid, D-malic acid, DL-malic acid, maleic acid or succinic acid. It also discloses saxagliptin phosphate hemihydrate (form A) and saxagliptin phosphate higher hydrate (form B). WO20111140328 discloses new polymorphs of saxagliptin and process for their preparation as well as additional processes to prepare saxagliptin monohydrate and saxagliptin hemihydrate. Finally, WO2012047871A1 discloses several crystal forms of saxagliptin hydrochloride and some saxagliptin hydrochloride forms as not crystalline pure forms.

It is well known that saxagliptin is prone to undergo an intra-molecular cyclization reaction in solution and solid states to form the corresponding cyclic amidine. The tablet formulation is prepared using an active coating process to minimize this formation. Saxagliptin is embedded within a film coat of Opadry spray coated onto inert core tablets. During the coating process, saxagliptin free base is converted in situ into hydrochloride salt. This reaction of degradation is described by G. S. Jones et al., in J. Org. Chem. 2011, vol. 76, pp. 10332-10337.

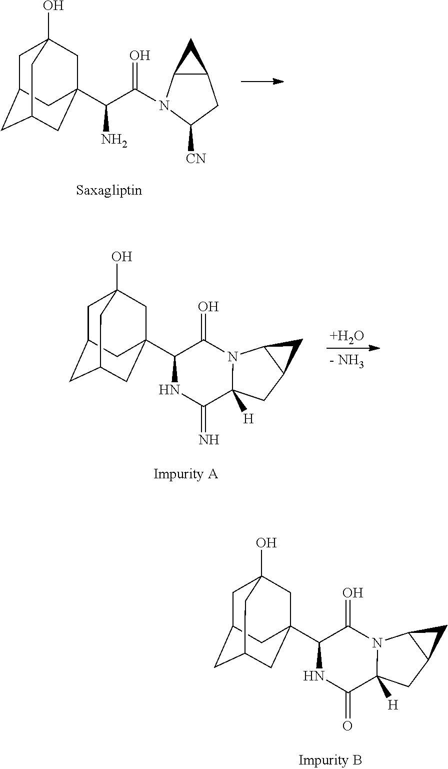

On the other hand, different solid forms of a pharmaceutically active ingredient can have different characteristics, and offer certain advantages, for example with regard to solubility or bioavailability. Thus, the discovery of new solid forms allows for improving the characteristics of the pharmaceutical formulations of the active ingredients, since some forms are more adequate for one type of formulation, and other forms for other different formulations. Furthermore, depending on the therapeutic indications, one or another pharmaceutical formulation may be preferred. However, the formulation is limited to a selected list of coformers and a special method of formulation to avoid degradation of saxagliptin.

Therefore, it is hence of interest to have new solid forms of saxagliptin or its salts.

### SUMMARY OF THE INVENTION

Inventors have found that saxagliptin hydrochloride can form cocrystals with several compounds, in particular, saxagliptin hydrochloride is able to form cocrystals with saxagliptin free base, glycolic acid, malonic acid, and urea.

The cocrystals have a specific stoichiometry which depends upon the structure of the second component. The provision of cocrystals of saxagliptin hydrochloride with a second component is considered a contribution to the art since the formation of different cocrystals can allow modulating stability, solubility, and hygroscopicity, and can provide enhanced bioavailability, enhanced galenic properties, or even higher purity.

While active pharmaceutical ingredients (APIs) have been recognized to form crystalline polymorphs, solvates, hydrates and amorphous forms, for a number of years, there is little knowledge about which ones can form cocrystals. By cocrystallizing an API or a salt of an API with a coformer (the second component of the cocrystal), a new solid state form of the API is created having unique properties compared with existing solid forms of the API or its salts. However, cocrystal formation is not predictable, and in fact is not always possible. Moreover, there is no way to predict the properties of a particular cocrystal of a compound until it is formed. Finding the right conditions to obtain a particular cocrystal can take significant time, effort, and resources. In the particular case of saxagliptin, there is neither a suggestion in the prior art regarding the possibility that saxagliptin hydrochloride could form cocrystals with a second component, nor an indication about how to prepare any possible cocrystal of saxagliptin.

Since different solid forms can exhibit different physicochemical profiles, the provision of cocrystals gives a new tool to overcome the problems associated with suboptimal properties of the known forms of saxagliptin or its salts. Cocrystals may provide an improvement of pharmacokinetic properties; an improvement of pharmaceutical properties, among others stability, hygroscopicity, flowability and compressibility. In addition, cocrystals have been demonstrated to be complementary or, at least, equal to salts for purification purposes, so products can be isolated using cocrystallization processes in order to obtain the desired specifications.

From a technical perspective, it is clear that cocrystal technology is able to solve problems that are difficult to tackle using more classical techniques. On occasion, they can provide a more straightforward solution, or simply the only solution, to a variety of problems. In addition, although a number of compounds are prone to form cocrystals, the conditions to obtain such cocrystals tend to be tricky and non-obvious.

Accordingly, an aspect of the present invention relates to the provision of a cocrystal of saxagliptin hydrochloride and a compound selected from the group consisting of saxagliptin, glycolic acid, malonic acid, and urea; or a solvate thereof, or a hydrate thereof. The cocrystals of the invention have, among other properties, enhanced chemical stability (avoiding the formation of amidine) and/or enhanced crystal stability (crystalline forms less prone to transform to H2-1).

The cocrystals of the invention may exist in solvated or unsolvated forms, including hydrated forms. It is to be understood that the invention encompasses all such solvated, as well as unsolvated forms. The obtention of solvates and hydrates depends on the solvent used and the crystallization conditions that can be determined by the skilled person. The cocrystals of the invention have a low amount of water (less than 2 molecules of water which is the amount of water of the form H2-1).

Saxagliptin hydrochloride which forms part of the cocrystals of the invention is a well-known drug useful for the treatment of type 2 diabetes. Accordingly, another aspect of the present invention relates to the provision of a cocrystal of saxagliptin hydrochloride and a compound selected from the group consisting of saxagliptin, glycolic acid, malonic acid, and urea; or a solvate thereof, or a hydrate thereof, for use as a medicament.

Another aspect of the present invention relates to the provision of a cocrystal of saxagliptin hydrochloride and a compound selected from the group consisting of saxagliptin, glycolic acid, malonic acid, and urea; or a solvate thereof, or a hydrate thereof, for use in the prevention and/or treatment of type 2 diabetes.

Another aspect of the present invention relates to the provision of a pharmaceutical composition comprising a cocrystal of saxagliptin hydrochloride and a compound selected from the group consisting of saxagliptin, glycolic acid, malonic acid, and urea; or a solvate thereof, or a hydrate thereof, together with appropriate amounts of pharmaceutical excipients or carriers. These pharmaceutical formulations can be produced by standard procedures known to the skilled person.

Another aspect of the present invention, relates to the provision of processes for the preparation of the cocrystals of the invention.

A further aspect of the present invention relates to the provision of a salt of saxagliptin with glycolic acid (1:1) hydrate. This salt has enhanced chemical stability, reducing the formation of amidine impurity. In particular, it relates to the provision of a salt of saxagliptin with glycolic acid (1:1) hydrate characterized by having an X-ray diffractogram that comprises characteristic peaks at approximately 7.2, 9.4, 10.9, 14.5, 15.1, 15.5, 17.6, 17.9, 18.7, 20.4, 21.8, 25.8 and 26.0 degrees 2 theta (Cu—K_{α} radiation, λ=1.5406 Å).

Another further aspect of the invention relates to the provision of a process for the preparation of the salt of saxagliptin with glycolic acid (1:1) hydrate as defined above.

Another further aspect of the invention, relates to the provision of the salt of saxagliptin with glycolic acid (1:1) hydrate as defined above, for use as a medicament.

Another further aspect of the invention relates to the provision of the salt of saxagliptin with glycolic acid (1:1) hydrate as defined above, for use in the prevention and/or treatment of type 2 diabetes.

Finally, a further aspect of the present invention relates to a pharmaceutical composition comprising the salt of saxagliptin with glycolic acid (1:1) hydrate as defined above, together with appropriate amounts of pharmaceutical excipients or carriers.

### BRIEF DESCRIPTION OF THE DRAWINGS

FIG. 1 shows the X-ray powder diffractogram (XRPD) of saxagliptin HCl.saxagliptin cocrystal (1:1) Form I.

FIG. 2 shows the XRPD of saxagliptin HCl glycolic acid cocrystal (1:1) hydrate Form II.

FIG. 3 shows the XRPD of saxagliptin HCl.malonic acid cocrystal (3:2) hydrate Form III.

FIG. 4 shows the XRPD of saxagliptin HCl.urea cocrystal (1:3) Form IV.

FIG. 5 shows the XRPD of a salt of saxagliptin.glycolic acid (1:1) hydrate Form A.

### DETAILED DESCRIPTION OF THE INVENTION

### Definitions

The term “cocrystal” refers herein to a crystalline entity with at least two different components constituting the unit cell at room temperature (20-25° C.) and interacting by weak interactions. Thus, in a cocrystal the active pharmaceutical ingredient crystallizes with one or more neutral components. The cocrystals may include one or more solvent molecules in the crystal lattice.

The term “weak interaction” refers herein as an interaction which is neither ionic nor covalent, and includes for example: hydrogen bonds, van der Waals interactions, and π-π stacking.

The term “solvate” is to be understood as meaning any form of the cocrystal in which the compound has attached to it via non-covalent binding solvent molecules. When the solvent is water the solvate is a hydrate.

A salt of saxagliptin refers herein to saxagliptin bound to another compound forming a salt by means of ionic interactions.

When a ratio of components of the cocrystals of the invention is specified it refers to the molar ratio between saxagliptin hydrochloride and a further component that forms the cocrystal.

The term “molar ratio” has been used to express the stoichiometric amount in mols of each of the components of a cocrystal.

When values of characteristic peaks of an X-ray diffractogram are given it is said that are “approximate” values. It should be understood that the values are the ones shown in the corresponding lists or tables ±0.1 degrees 2 theta measured in an X-ray diffractometer with Cu—K_{α} radiation λ=1.5406 Å.

The term “room temperature” as disclosed herein refers to a temperature of the environment, without heating or cooling, and is generally comprised of from 20 to 25° C.

For the purposes of the invention, any ranges given include both the lower and the upper end-points of the range. Ranges given, such as temperatures, times, and the like, should be considered approximate, unless specifically stated.

Unless otherwise stated, the expression “substantially free of other crystalline forms” is to be understood to mean that the cocrystals of the invention show an XRPD with the characteristic peaks mentioned below for each cocrystal but without having any other significant peak of a subject compound which is an organic or inorganic compound in crystalline form. The subject compound may be other crystalline form of saxagliptin or a salt thereof or an inorganic salt. Examples of inorganic salts include sodium chloride, potassium chloride or ammonium chloride.

By the expression “without any other significant peak” is understood that there is not any other peak with a relative intensity equal to or higher than 3% corresponding to a subject compound as measured by XRPD, preferably, 1% or higher, more preferably, 0.5% or higher, even more preferably, 0.1% or higher, still even more preferably is free of a subject compound.

The expression “cocrystal obtainable by” is used here to define each specific cocrystal of the invention by the process for obtaining it and refers to the product obtainable by any of the corresponding processes disclosed herein. For the purposes of the invention the expressions “obtainable”, “obtained” and equivalent expressions are used interchangeably, and in any case, the expression “obtainable” encompasses the expression “obtained”.

The terms “wet grinding” and “liquid assisted grinding” are equivalent and refer to a technique which consists of milling or grinding the product or mixture with some drops of solvent added. Neat and liquid-assisted grinding are techniques that can be employed in order to produce cocrystals. In neat (dry) grinding, cocrystal formers are ground together manually using a mortar and pestle, using a ball mill, or using a vibratory mill. In liquid-assisted grinding, or kneading, a small or substoichiometric amount of liquid (solvent) is added to the grinding mixture.

The term “therapeutically effective amount” as used herein, refers to the amount of a compound that, when administered, is sufficient to prevent development of, or alleviate to some extent, one or more of the symptoms of the illness to be treated. The particular dose of compound administered according to this invention will of course be determined by the particular circumstances surrounding the case, including the compound administered, the route of administration, the particular condition being treated, and the similar considerations.

The term “pharmaceutical composition” refers to a mixture of a compound disclosed herein with other chemical components, such as diluents or carriers.

The pharmaceutical composition facilitates administration of the compound to an organism.

The term “pharmaceutically acceptable excipients or carriers” refers to pharmaceutically acceptable material, composition or vehicle, such as liquid or solid filler, diluent, excipient, solvent, or encapsulating material. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio.

The term “treatment” meant to include alleviating or eradicating a disorder, disease, or condition, or one or more of the symptoms associated with the disorder, disease or condition, or alleviating or eradicating the cause(s) of the disorder, disease, or condition itself.

The term “ethyl acetate saturated with water” refers to ethyl acetate having dissolved an amount of water, as a result of mixing the ethyl acetate with a considerably amount of water, followed by stopping the stirring to allow the mixture to be separated into two phases, and discharging the aqueous phase.

Generally, the water content is comprised of from 3% to 4% w/w at 20° C. In particular, up to 3.3 ml of water can be dissolved in 90 ml of anhydrous ethyl acetate at 20° C.

The saxagliptin monohydrate cited in the present application and which has been used as starting material of several processes disclosed herein to obtain the cocrystals of the invention, corresponds to the form named as Form H-1 in WO2008131149. The saxagliptin hydrochloride dihydrate cited in the present application and which has been used as starting material of several processes disclosed herein to obtain the cocrystals of the invention, corresponds to the Form named H2-1 in WO2008131149.

As mentioned above, it is provided a cocrystal of saxagliptin hydrochloride and a compound selected from the group consisting of saxagliptin, glycolic acid, malonic acid, and urea; or a solvate thereof, or a hydrate thereof.

Preferred cocrystals, as well as preferred crystalline forms thereof are detailed below.

In a preferred embodiment, any of the cocrystals of the invention is substantially free of any other crystalline forms. The other crystalline form may be an organic or an inorganic compound.

In a more preferred embodiment, any of the cocrystals of the invention is substantially free of any other crystalline form comprising saxagliptin or a salt thereof.

In another more preferred embodiment, any of the cocrystals of the invention is substantially free of any inorganic salt in crystalline form.

Preferred cocrystals are those which can be readily prepared, easy to scale-up, have acceptable shelf-life and are in form accepted for use in pharmaceutical formulations.

Each of the crystalline forms of the cocrystals of the present invention has been characterized at least by XRPD. Proton nuclear magnetic resonance analyses (¹H NMR), in particular, ¹H NMR (DMSO-d₆) and/or ¹H NMR (MeOD-d₄) may have also been included.

The XRPD analysis disclosed herein had been performed using a PANalytical X'Pert diffractometer with CuKₐ radiation (λ=1.5406 Å) in Bragg-Brentano geometry. The system is equipped with a monodimensional, real time multiple strip detector. The diffractograms were recorded from 3° to 40° at a scan rate of 17.6° per minute.

The ¹H NMR (DMSO-d₆) disclosed herein had been recorded in deuterated dimethyl sulfoxide (DMSO-d₆) in a Varian Mercury 400 spectrometer, equipped with a broadband probe ATB 1H/19F/X of 5 mm. Spectra were acquired dissolving 5-10 mg of sample in 0.6 mL of deuterated solvent.

The ¹H NMR (MeOD-d₄) disclosed herein had been recorded in deuterated methanol-d₄ (MeOD-d₄) in a Varian Mercury 400 spectrometer, equipped with a broadband probe ATB 1H/19F/X of 5 mm. Spectra were acquired dissolving 5-10 mg of sample in 0.6 mL of deuterated solvent.

Thermogravimetric analyses were recorded in a thermogravimetric analyzer Mettler TGA/SDTA851e. A sample between 3.7 mg and 5.8 mg was weighed into a 70 μL alumina crucible with a pinhole lid and was heated at 10° C./min from 30 to 300° C., under nitrogen (50 mL/min).

Cocrystals of saxagliptin hydrochloride and a compound selected from saxagliptin, glycolic acid, malonic acid, or urea; or a solvate thereof, or a hydrate thereof, can have different molar ratios between the saxagliptin hydrochloride and the further component.

In a preferred embodiment of the invention, the cocrystal is saxagliptin HCl.saxagliptin cocrystal hydrate in a molar ratio 1:1. The ratio of saxagliptin HCl/saxagliptin in the saxagliptin HCl.saxagliptin cocrystal may be determined by titration as it is disclosed in Example 1.

Depending on the drying conditions, there are small variations on the position of some diffraction peaks, being the most representative the variation of the peak at 8.6. Depending on the hydration grade this peak can be between 8.3 and 8.7 degrees 2 theta (Cu—K_{α} radiation, λ=1.5406 Å). The cocrystals of saxagliptin HCl.saxagliptin cocrystal (1:1) hydrate are characterized by having an X-ray diffractogram that comprises a characteristic peak between 8.3 and 8.7 degrees 2 theta and other characteristic peaks at approximately 6.8, 13.6, 14.3, 15.1, and 19.1 degrees 2 theta (Cu—K_{α} radiation, λ=1.5406 Å). They may have an amount of water between 0.1 and 5% w/w measured by TGA.

In a more preferred embodiment, the cocrystal is a saxagliptin HCl.saxagliptin cocrystal (1:1) hydrate, named herein cocrystal Form I, which is characterized by having an X-ray diffractogram that comprises characteristic peaks at approximately 6.8, 13.6, 14.3, 15.1, and 19.1 degrees 2 theta (Cu—K_{α} radiation, λ=1.5406 Å). In a still more preferred embodiment, the cocrystal of saxagliptin HCl.saxagliptin cocrystal (1:1) hydrate is characterized by having an X-ray diffractogram that comprises characteristic peaks at approximately 6.8, 8.0, 8.6, 13.6, 14.3, 15.1, 16.9, 18.5, 19.1, 22.6, 27.8, and 29.8 degrees 2 theta (Cu—K_{α} radiation, λ=1.5406 Å). In an even still more preferred embodiment, the cocrystal of saxagliptin HCl.saxagliptin cocrystal (1:1) hydrate is characterized by having an X-ray diffractogram that comprises characteristic peaks at approximately 6.8, 8.0, 8.6, 13.6, 14.3, 15.1, 16.9, 17.2, 17.5, 17.6, 18.5, 19.1, 22.6, 27.8, and 29.8 degrees 2 theta (Cu—K_{α} radiation, λ=1.5406 Å). More specifically, this new cocrystal Form I is characterized by exhibiting in the powder X-ray diffractogram a pattern of peaks, expressed in 2 theta units in degrees, 2θ (°), which is shown in Table 1.

TABLE 1
List of selected peaks of XRPD (only peaks with relative intensity
greater than or equal to 1% are indicated):

| Pos. | Rel. Int. |
|---|---|
| [°2Th.] | [%] |
| 6.8 | 100 |
| 8.0 | 5 |
| 8.6 | 15 |
| 11.1 | 2 |
| 13.6 | 57 |
| 14.3 | 45 |
| 14.5 | 4 |
| 15.1 | 22 |
| 15.7 | 2 |
| 16.9 | 8 |
| 17.2 | 13 |
| 17.5 | 17 |
| 17.6 | 12 |
| 18.2 | 4 |
| 18.5 | 20 |
| 19.1 | 44 |
| 20.6 | 3 |
| 20.9 | 2 |
| 21.4 | 4 |
| 21.8 | 5 |
| 22.6 | 9 |
| 24.3 | 8 |
| 25.0 | 6 |
| 25.6 | 7 |
| 26.0 | 2 |
| 27.2 | 1 |
| 27.5 | 3 |
| 27.8 | 13 |
| 29.1 | 1 |
| 29.8 | 6 |
| 30.5 | 2 |
| 31.3 | 2 |
| 32.2 | 5 |
| 33.3 | 1 |
| 34.5 | 2 |
| 34.8 | 3 |
| 35.7 | 3 |
| 37.2 | 1 |

Even more specifically, this cocrystal Form I is characterized by having an X-ray diffractogram that comprises characteristic peaks at approximately 6.8, 8.0, 8.6, 11.1, 13.6, 14.3, 14.5, 15.1, 15.7, 16.9, 17.2, 17.5, 17.6, 18.2, 18.5, 19.1, 20.6, 20.9, 21.4, 21.8, 22.6, 24.3, 25.0, 25.6, 26.0, 27.2, 27.5, 27.8, 29.1, 29.8, 30.5, 31.3, 32.2, 33.3, 34.5, 34.8, 35.7, and 37.2 degrees 2 theta (Cu—K_{α} radiation, λ=1.5406 Å) and without having any significant peak of other crystalline form, in particular, of any other crystalline form of saxagliptin or a salt thereof or of an inorganic salt.

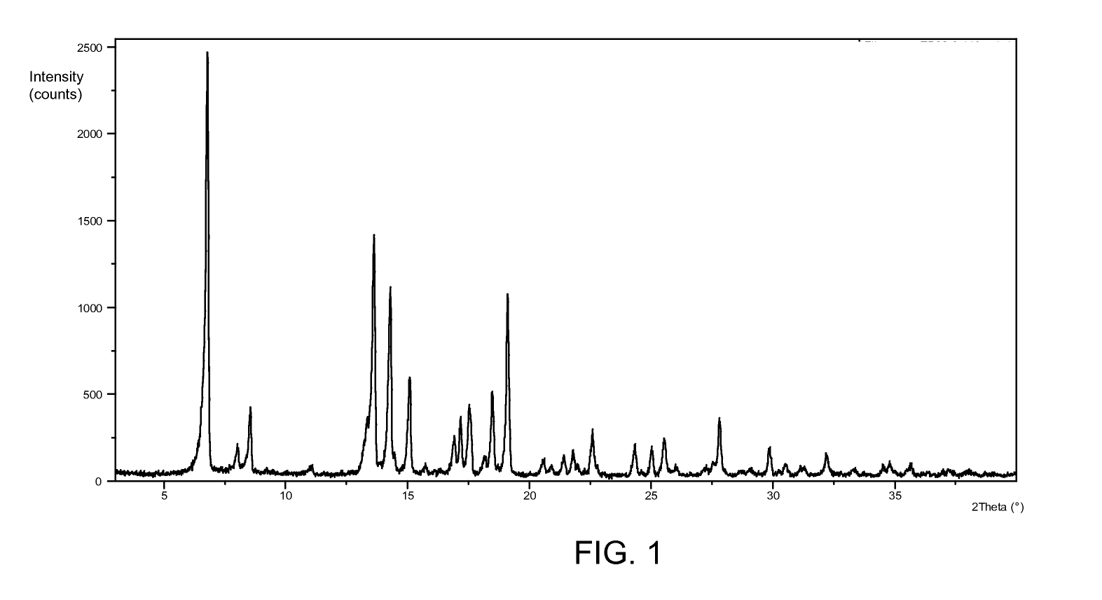

This cocrystal Form I may be further characterized by an X-ray diffractogram as in FIG. 1.

This cocrystal Form I may also be further characterized by the following ¹H NMR spectra ¹H NMR (DMSO-d₆, 400 MHz): δ=5.14 (d, J=10.6 Hz, 1 H); 5.03 (s br, 2H); 4.45 (s, 1H); 4.01-3.91 (m, 1H); 3.74 (s, 1H); 2.21 (dd, J=1.2 Hz, J=13.7 Hz, 1H); 2.12 (s, 2H); 1.94-1.83 (m, 1H); 1.75-1.28 (m, 12H); 1.05-0.94 (m, 1H); 0.78-0.67 (m, 1H), or ¹H NMR (MeOD-d₄, 400 MHz): 6=5.12 (dd, J=10.6 Hz, J=10.6 Hz, 1H); 3.90-3.82 (m, 1H); 3.85 (s, 1H); 2.61 (ddd, J=5.9 Hz, J=10.6 Hz, J=13.7 Hz, 1H); 2.32 (dd, J=2.4 Hz, J=13.7 Hz, 1H); 2.24 (s, 2H); 2.02-1.91 (m, 1H); 1.84-1.48 (m, 13H); 1.13-1.04 (m, 1H); 0.99-0.92 (m, 1H).

The cocrystal Form I generally have an amount of water between 2.5 and 3% w/w measured by TGA, although it could also vary between 0.1 and 5% w/w. The saxagliptin HCl.saxagliptin cocrystal (1:1) hydrate Form I defined above has advantageous properties to be used in the pharmaceutical field. It is very crystalline, it is soluble in water, and it shows chemical stability. As it is illustrated in the Examples, it is clear that the degradation rate is much lower for the cocrystal Form I than for the saxagliptin free base H-1 and saxagliptin hydrochloride dihydrate H2-1 used as comparative Examples. Thus, it has enhanced chemical stability, reducing the formation of amidine impurity.

The saxagliptin HCl.saxagliptin cocrystal (1:1) hydrate Form I as defined above may be prepared by a process which comprises the following steps: (a) wet grinding of a mixture of saxagliptin monohydrate H-1 form and saxagliptin hydrochloride dihydrate H2-1 form in isopropanol; and (b) isolating the compound thus obtained.

Alternatively, the saxagliptin HCl.saxagliptin cocrystal (1:1) hydrate Form I as defined above may also be prepared by a process which comprises the following steps: (a) wet grinding of a mixture of saxagliptin monohydrate Form H-1 and saxagliptin hydrochloride dihydrate Form H2-1 in ethyl acetate saturated with water; and (b) isolating the compound thus obtained. Generally, the wet grinding is carried out at room temperature.

The saxagliptin HCl.saxagliptin cocrystal (1:1) hydrate Form I defined above can also be obtained by crystallization from a solution of saxagliptin free base in a mixture of a (C₁-C₆)-alcohol and water as solvent. It can also be obtained by crystallization from a solution of saxagliptin free base in methylisobutylketone or ethyl acetate. Generally, the crystallization occurs after adding 0.25 to 0.6 eq. hydrochloric acid, preferably 0.5 eq.

Preferably, the saxagliptin HCl.saxagliptin cocrystal (1:1) hydrate Form I defined above is obtained by providing a solution of saxagliptin in a solvent system selected from the group consisting of water/(C₂-C₃)-alcohol, (C₂-C₃)-alcohol, methylisobutylketone, and ethyl acetate; adding a solution of hydrochloric acid in water or water/(C₂-C₃)-alcohol; and subsequently isolating the precipitated compound thus obtained.

In a preferred embodiment, the (C₂-C₃)-alcohol is ethanol or isopropanol. In a more preferred embodiment, the solvent is isopropanol.

The hydrochloric acid may be added in a certain period of time, for instance, in about 30′.

Preferably, the solution is seeded to start the crystallization with saxagliptin HCl.saxagliptin (1:1) cocrystal Form I. In a more preferred embodiment, the mixture is seeded with cocrystal Form I at the beginning of the addition of the hydrochloric acid and when a half of the hydrochloric acid solution has been added. The seeding cocrystal form may be obtained by any of the processes described above.

The cocrystal formed by this method may be separated from the medium at room temperature or at a lower temperature, for instance, the mixture may be cooled at a temperature about 0-5° C. before separating the product. The cocrystal formed may be separated by filtration or other suitable techniques as known to a skilled person in the art.

In a particular embodiment of the process, the process further comprises a previous step of mixing saxagliptin monohydrate with a solvent system selected from the group consisting of water/(C₁-C₆)-alcohol, (C₁-C₆)-alcohol, methylisobutylketone, and ethyl acetate, to yield a solution of saxagliptin in the corresponding solvent. In a preferred embodiment, the (C₁-C₆)-alcohol is ethanol or isopropanol. In a more preferred embodiment, the solvent is isopropanol.

This process is appropriate to be used at industrial scale and uses solvents with a low toxicity.

The compound isolated in any of the previous processes can be dried at room temperature, preferably under vacuum. Generally, the vacuum is comprised of 0.5 to 3 mbar.

Generally, the molar ratio of saxagliptin HCl and saxagliptin starting materials of each of the previous processes is 1:1, although a small excess of any of them can be used depending on the obtention process.

The stoichiometry is confirmed by titration of a cocrystal of saxagliptin HCl and saxagliptin obtained by crystallization as it is illustrated in the Examples.

The saxagliptin HCl.saxagliptin cocrystal (1:1) hydrate Form I of the invention may also be defined by its preparation process. Accordingly, this aspect of the invention can be formulated as saxagliptin HCl.saxagliptin (1:1) hydrate cocrystal Form I as defined above, obtainable by any of the previous processes, optionally including any preferred or particular embodiment of the process and possible combinations of some of the process features disclosed above.

In another preferred embodiment, the cocrystal is a saxagliptin HCl glycolic acid cocrystal in the form of a hydrate in a ratio of saxagliptin HCl glycolic acid of 1:1.

Preferably, the cocrystal is saxagliptin HCl glycolic acid cocrystal (1:1) monohydrate, named herein cocrystal Form II, which is characterized by having an X-ray diffractogram that comprises characteristic peaks at approximately 13.0, 14.4, 18.3, 18.9, 22.3 and 25.8 degrees 2 theta (Cu—K_{α} radiation, λ=1.5406 Å). More preferably, it is characterized by having an X-ray diffractogram that comprises characteristic peaks at approximately 6.5, 13.0, 13.8, 14.4, 17.0, 18.1, 18.3, 18.9, 19.5, 22.3, 23.4, 23.6, 25.8 and 26.6 degrees 2 theta (Cu—K_{α} radiation, λ=1.5406 Å). Even more preferably, it is characterized by having an X-ray diffractogram that comprises characteristic peaks at approximately 6.5, 11.1, 13.0, 13.8, 14.4, 15.1, 15.8, 16.4, 17.0, 18.1, 18.3, 18.9, 19.5, 20.4, 22.3, 23.4, 23.6, 25.8, 26.6, 27.5, 27.9, 28.8, 31.7, 33.7, and 34.3 degrees 2 theta (Cu—K_{α} radiation, λ=1.5406 Å).

More specifically, this cocrystal Form II exhibits in the powder X-ray diffractogram a pattern of peaks, expressed in 2 theta units in degrees, 20)(°, which is shown in Table 2.

TABLE 2
List of selected peaks of XRPD (only peaks with relative intensity
greater than or equal to 1% are indicated):

| Pos. | Rel. Int. |
|---|---|
| [°2Th.] | [%] |
| 6.5 | 40 |
| 9.1 | 6 |
| 11.1 | 11 |
| 13.0 | 54 |
| 13.8 | 27 |
| 14.4 | 100 |
| 15.1 | 15 |
| 15.8 | 10 |
| 16.4 | 10 |
| 17.0 | 21 |
| 18.1 | 89 |
| 18.3 | 70 |
| 18.9 | 67 |
| 19.5 | 30 |
| 20.4 | 15 |
| 22.3 | 89 |
| 23.4 | 24 |
| 23.6 | 23 |
| 25.8 | 39 |
| 26.6 | 32 |
| 27.0 | 6 |
| 27.5 | 14 |
| 27.9 | 18 |
| 28.8 | 19 |
| 29.4 | 9 |
| 30.6 | 8 |
| 31.7 | 10 |
| 32.1 | 5 |
| 33.2 | 7 |
| 33.7 | 15 |
| 34.3 | 11 |
| 34.8 | 8 |
| 35.3 | 5 |
| 36.2 | 7 |
| 36.8 | 7 |
| 37.6 | 3 |
| 38.3 | 2 |
| 39.0 | 9 |

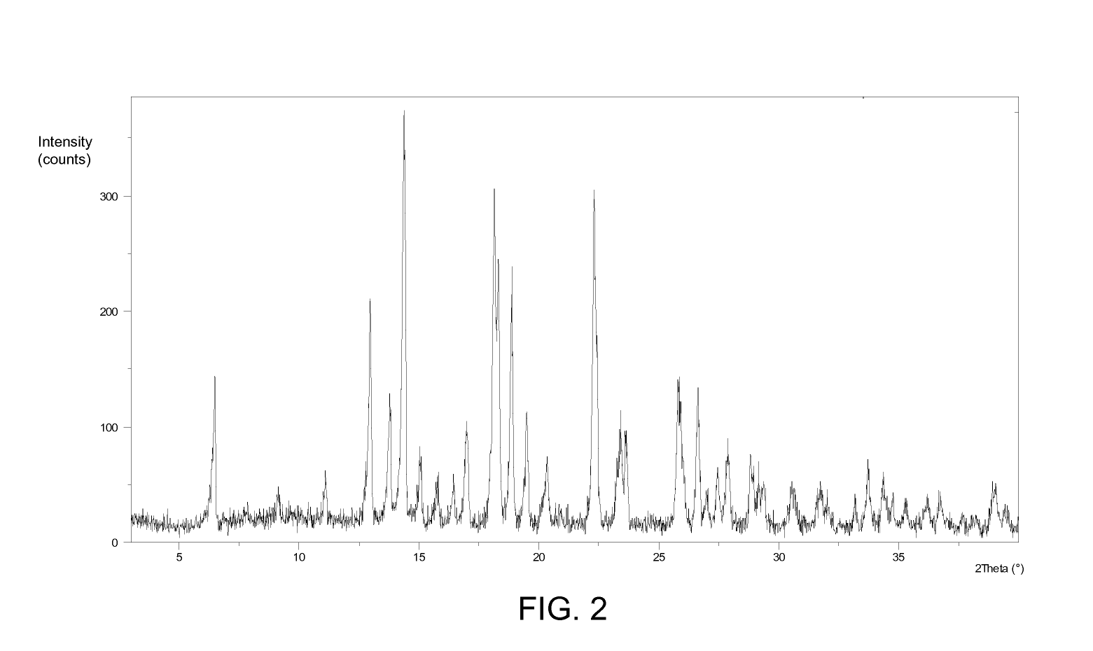

This cocrystal Form II may be further characterized by an X-ray diffractogram as in FIG. 2.

The cocrystal Form II may also be further characterized by the following ¹H NMR spectrum (DMSO-d₆, 400 MHz): δ=5.24 (dd, J=1.2 Hz, J=10.6 Hz, 1H); 4.60 (s, 1H); 4.23 (s, 1H); 4.14-4.05 (m, 1H); 3.90 (s, 2H); 2.26 (d, J=14.1 Hz, 1H); 2.16 (s, 2H); 2.02-1.90 (m, 1H); 1.73-1.34 (m, 12H); 1.08-0.97 (m, 1H); 0.82-0.71 (m, 1H).

The saxagliptin HCl glycolic acid cocrystal (1:1) monohydrate Form II defined above has the following advantageous properties to be used in the pharmaceutical field: It is quite soluble in water (5 volumes of water at room temperature), and the amount of water in the cocrystal is well defined, corresponding to a monohydrate.

The saxagliptin HCl glycolic acid cocrystal (1:1) monohydrate Form II may be prepared by a process which comprises (a) suspending saxagliptin hydrochloride dihydrate Form H2-1 and glycolic acid in a (C₆-C₈)-aromatic hydrocarbon such as toluene or xylene, during the necessary period of time for the conversion of the starting materials into the saxagliptin HCl glycolic acid cocrystal (1:1) monohydrate be produced; and (b) isolating the compound thus obtained. The step (a) may be carried out at room temperature. Preferably, the (C₆-C₈)-aromatic hydrocarbon is xylene. Generally the suspension is stirred at room temperature during at least 12 hours. The isolation in step (b) can be carried out by filtration or centrifugation. The cocrystal thus obtained can be dried, preferably at room temperature and under vacuum. Generally, the vacuum is comprised of 0.5 to 3 mbar.

Generally, the molar ratio of saxagliptin hydrochloride dihydrate and glycolic acid starting materials in the previous process is 1:1. A small excess of any of them could be used depending on the obtention process. The saxagliptin HCl glycolic acid cocrystal (1:1) monohydrate Form II may also be defined by its preparation process. Accordingly, this aspect of the invention can be formulated as saxagliptin HCl glycolic acid cocrystal (1:1) monohydrate Form II as defined above, obtainable by the previous process optionally including any preferred or particular embodiment of the process and possible combinations of some of the process features disclosed above.

In another preferred embodiment, the cocrystal is a saxagliptin HCl malonic acid cocrystal in a ratio of saxagliptin HCl.malonic acid of from 1:1 to 2:1. In a more preferred embodiment, the cocrystal is saxagliptin HCl.malonic acid cocrystal (3:2) hydrate.

In a still more preferred embodiment, the cocrystal is a saxagliptin HCl.malonic acid cocrystal (3:2) hydrate, named herein cocrystal Form III, which is characterized by having an X-ray diffractogram that comprises characteristic peaks at approximately 6.3, 14.4, 18.2, 22.4 and 25.8 degrees 2 theta (Cu—K_{α} radiation, λ=1.5406 Å).

More preferably, it is characterized by having an X-ray diffractogram that comprises characteristic peaks at approximately 6.3, 8.0, 12.6, 13.5, 14.4, 17.1, 18.2, 18.4, 18.7, 19.0, 22.4, 23.1 and 25.8 degrees 2 theta (Cu—K_{α} radiation, λ=1.5406 Å). Even more preferably, it is characterized by having an X-ray diffractogram that comprises characteristic peaks at approximately 6.3, 8.0, 9.1, 12.6, 13.5, 14.4, 15.2, 16.0, 17.1, 18.2, 18.4, 18.7, 19.0, 20.6, 22.2, 22.4, 23.1, 25.8, 28.1, 28.7, 29.5, and 34.0 degrees 2 theta (Cu—K_{α} radiation, λ=1.5406 Å).

More specifically, this cocrystal Form III is characterized by exhibiting in the powder X-ray diffractogram a pattern of peaks, expressed in 2 theta units in degrees, 2θ (°), which is shown in Table 3.

TABLE 3
List of selected peaks of XRPD (only peaks with relative intensity
greater than or equal to 1% are indicated):

| Pos. | Rel. Int. |
|---|---|
| [°2Th.] | [%] |
| 6.3 | 57 |
| 8.0 | 17 |
| 9.1 | 17 |
| 12.6 | 50 |
| 13.5 | 57 |
| 14.4 | 100 |
| 15.2 | 17 |
| 16.0 | 13 |
| 16.3 | 10 |
| 17.1 | 25 |
| 18.2 | 61 |
| 18.4 | 55 |
| 18.7 | 50 |
| 19.0 | 38 |
| 20.6 | 11 |
| 22.2 | 25 |
| 22.4 | 60 |
| 23.1 | 25 |
| 24.1 | 10 |
| 24.8 | 6 |
| 25.8 | 18 |
| 26.1 | 9 |
| 26.9 | 7 |
| 27.1 | 3 |
| 27.4 | 9 |
| 28.1 | 11 |
| 28.3 | 8 |
| 28.7 | 14 |
| 29.5 | 13 |
| 30.5 | 4 |
| 31.3 | 7 |
| 31.6 | 7 |
| 32.5 | 4 |
| 33.0 | 4 |
| 34.0 | 14 |
| 36.5 | 5 |
| 38.9 | 4 |

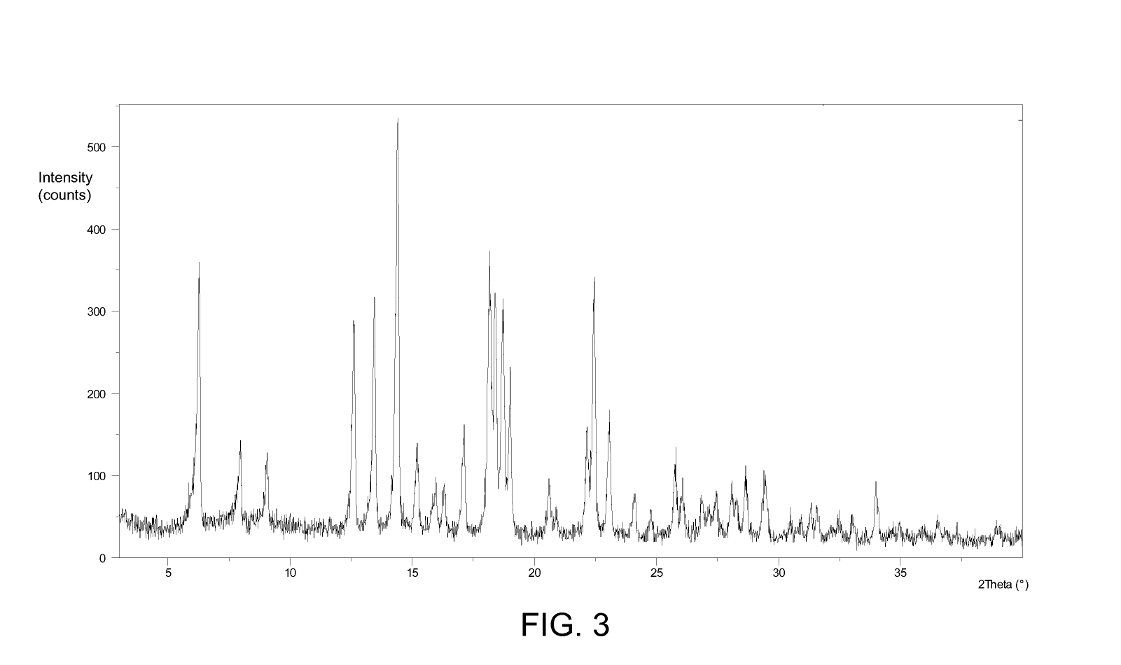

This cocrystal Form III may be further characterized by an X-ray diffractogram as in FIG. 3.

The cocrystal Form III may also be further characterized by the following ¹H NMR spectrum (DMSO-d₆, 400 MHz): δ=8.23 (s br, 3H); 5.23 (dd, J=2.0 Hz, J=10.6 Hz, 1H); 4.60 (s br, 1H); 4.23 (s, 1H); 4.14-4.06 (m, 1H); 3.24 (s, 1.6H); 2.25 (dd, J=2.3 Hz, J=14.1 Hz, 1H); 2.16 (s, 2H); 2.01-1.90 (m, 1H); 1.71-1.37 (m, 12H); 1.08-0.98 (m, 1H); 0.80-0.72 (m, 1H).

The cocrystal Form III generally has an amount of water between 0.1 and 3% w/w, more preferably between 2 and 2.5% w/w measured by TGA.

The saxagliptin HCl.malonic acid cocrystal (3:2) hydrate Form III defined above has advantageous properties to be used in the pharmaceutical field. It is very crystalline and it is soluble in water.

The saxagliptin HCl.malonic acid cocrystal (3:2) hydrate Form III may be prepared by a process comprising the steps of (a) wet grinding of saxagliptin hydrochloride dihydrate Form H2-1 and malonic acid in methanol generally at room temperature during the necessary period of time for the conversion be produced; and (b) isolating the compound thus obtained. Generally, the time needed in step (a) is comprised of from 30′ to 1 h. Preferably, this time is about 45′. The cocrystal thus obtained can be dried, preferably at room temperature and under vacuum. Generally, the vacuum is comprised of 0.5 to 3 mbar.

The saxagliptin HCl.malonic acid cocrystal (3:2) hydrate Form III may also be prepared by a process comprising the steps of (a) suspending saxagliptin monohydrate Form H-1 and malonic acid in a (C₆-C₈) aromatic hydrocarbon, at a temperature comprised of from 70-90° C., preferably at about 80° C.; (b) Optionally, seeding the suspension with saxagliptin HCl.malonic acid cocrystal (3:2) hydrate previously obtained, and adding a solution of hydrochloric acid in isopropanol; and (c) isolating the compound thus obtained.

Preferably, the (C₆-C₈) aromatic hydrocarbon is toluene. Preferably the temperature of step (a) is about 80° C. The seeding crystal may be obtained by any of the processes herein disclosed to prepare the saxagliptin HCl.malonic acid cocrystal (3:2) hydrate.

The isolation of step (c) can be carried out by cooling the mixture at room temperature and by separating the product from the mixture by filtration or centrifugation. The cocrystal thus obtained can be dried, preferably at room temperature and under vacuum. Generally, the vacuum is comprised of 0.5 to 3 mbar.

Generally, when the starting material is saxagliptin hydrochloride dihydrate, the molar ratio of saxagliptin hydrochloride dihydrate and malonic acid starting materials is 1:1, although a small excess of any of them can be used depending on the obtention process.

The saxagliptin HCl.malonic acid cocrystal (3:2) hydrate Form III may also be defined by its preparation process. Accordingly, this aspect of the invention can be formulated saxagliptin HCl.malonic acid cocrystal (3:2) hydrate form as defined above, obtainable by any of previous processes, optionally including any preferred or particular embodiment of the process and possible combinations of some of the process features disclosed above.

In another preferred embodiment, the cocrystal is saxagliptin HCl.urea cocrystal (1:3).

In a more preferred embodiment, the cocrystal is the saxagliptin HCl.urea cocrystal (1:3), named herein Form IV, which is characterized by having an X-ray diffractogram that comprises characteristic peaks at approximately 4.8, 9.7, 13.1, 17.8 and 20.4 degrees 2 theta (Cu—K_{α} radiation, λ=1.5406 Å). More preferably, it is characterized by having an X-ray diffractogram that comprises characteristic peaks at 4.8, 8.1, 9.7, 12.9, 13.1, 14.6, 15.4, 17.8, 20.4, and 21.1 degrees 2 theta (Cu—K_{α} radiation, λ=1.5406 Å).

More specifically, this new cocrystal Form IV is characterized by exhibiting in the powder X-ray diffractogram a pattern of peaks, expressed in 2 theta units in degrees, 2θ (°), which is shown in Table 4.

TABLE 4
List of selected peaks of XRPD (only peaks with relative intensity
greater than or equal to 1% are indicated):

| Pos. | Rel. Int. |
|---|---|
| [°2Th.] | [%] |
| 4.8 | 16 |
| 8.1 | 23 |
| 9.7 | 100 |
| 12.9 | 18 |
| 13.1 | 44 |
| 13.8 | 6 |
| 14.6 | 49 |
| 15.4 | 19 |
| 17.8 | 29 |
| 19.6 | 4 |
| 20.4 | 34 |
| 21.1 | 19 |
| 22.2 | 7 |
| 24.4 | 9 |
| 25.7 | 9 |
| 26.1 | 5 |
| 27.1 | 5 |
| 27.8 | 4 |

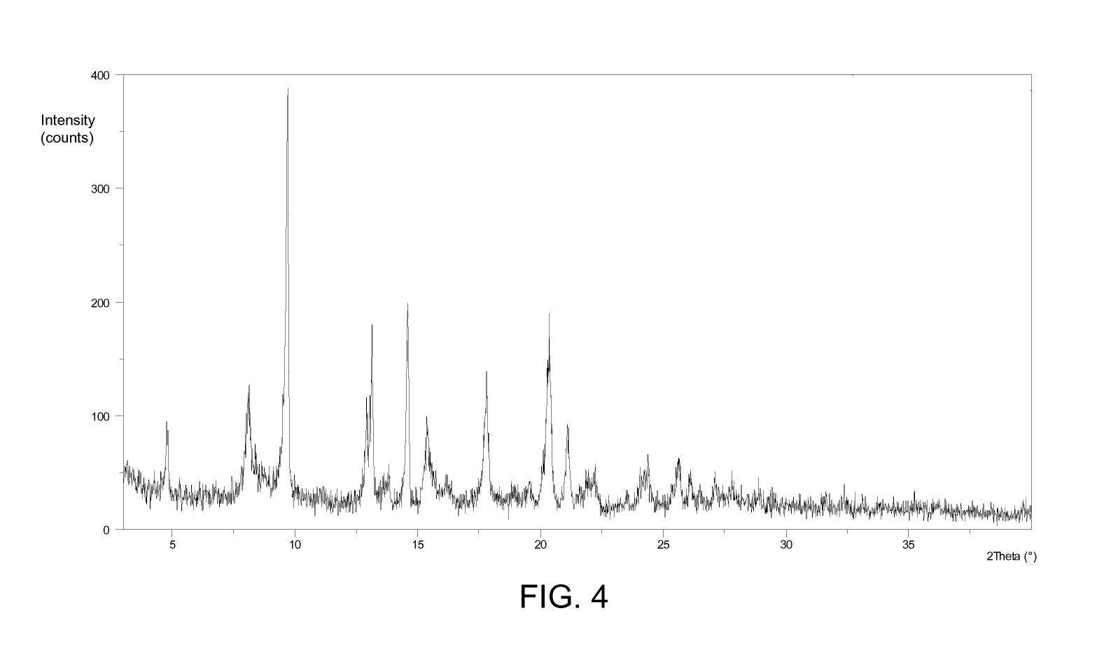

This cocrystal Form IV may be further characterized by an X-ray diffractogram as in FIG. 4.

The cocrystal Form IV may also be further characterized by the following ¹H NMR spectrum (DMSO-d₆, 400 MHz): δ=8.21 (s br, 3H); 5.41 (s br, 12H); 5.24 (dd, J=2.0 Hz, J=10.6 Hz, 1H); 4.60 (s, 1H); 4.23 (s, 1H); 4.13-4.06 (m, 1H); 2.26 (dd, J=2.0 Hz, J=14.1 Hz, 1H); 2.16 (s, 2H); 2.01-1.91 (m, 1H); 1.71-1.36 (m, 12H); 1.09-0.97 (m, 1H); 0.80-0.72 (m, 1H).

The saxagliptin HCl.urea cocrystal (1:3) Form IV defined above has advantageous properties to be used in the pharmaceutical field. It is very crystalline and it is very soluble in water. In addition, the amount of water present in the structure is very low, in particular, the amount of water is comprised of from 0.1 to 0.3 equivalents.

The saxagliptin HCl.urea cocrystal (1:3) Form IV may be prepared by a process which comprises: (a) suspending saxagliptin hydrochloride dihydrate Form H2-1 and urea in ethyl acetate at a temperature comprised of from 60-70° C.; and (b) isolating the compound thus obtained.

Preferably the temperature of step (a) is 65° C. In a particular embodiment, the isolation step (b) comprises cooling the mixture at room temperature and separating the product from the mixture by filtration or centrifugation. The cocrystal thus obtained can be dried, preferably at room temperature and under vacuum. Generally, the vacuum is comprised of 0.5 to 3 mbar.

The saxagliptin HCl.urea cocrystal (1:3) Form IV may also be defined by its preparation process. Accordingly, this aspect of the invention can be formulated as saxagliptin HCl.urea cocrystal (1:3) Form IV as defined above, obtainable by any of previous processes, optionally including any preferred or particular embodiment of the process and possible combinations of some of the process features disclosed above.

Generally, the molar ratio of saxagliptin HCl and urea starting materials of each of the previous processes is 1:3, although a small excess of any of them can be used depending on the obtention process.

It is also part of the invention a salt of saxagliptin with glycolic acid (1:1) hydrate, named herein Form A, which is characterized by having an X-ray diffractogram that comprises characteristic peaks at approximately 7.2, 10.9, 17.6, 17.9 and 21.8 degrees 2 theta (Cu—K_{α} radiation, λ=1.5406 Å).

Preferably, it is characterized by having an X-ray diffractogram that comprises characteristic peaks at approximately 7.2, 9.4, 10.9, 14.5, 15.1, 15.5, 17.6, 17.9, 18.7, 20.4, 21.8, 25.8 and 26.0 degrees 2 theta (Cu—K_{α} radiation, λ=1.5406 Å). More preferably, it is characterized by having an X-ray diffractogram that comprises characteristic peaks at approximately 7.2, 9.4, 10.9, 14.5, 14.7, 15.1, 15.5, 16.6, 17.2, 17.6, 17.9, 18.7, 19.6, 20.4, 21.5, 21.8, 22.2, 22.6, 25.8 and 26.0 degrees 2 theta.

This new salt Form A is characterized by exhibiting in the powder X-ray diffractogram a pattern of peaks, expressed in 2 theta units in degrees, 20)(°, which is shown in Table 5.

TABLE 5
List of selected peaks of XRPD (only peaks with relative intensity
greater than or equal to 1% are indicated):

| | Rel. |
|---|---|
| Pos. [°2Th.] | Int. [%] |
| 7.2 | 100 |
| 9.4 | 69 |
| 10.9 | 82 |
| 12.0 | 6 |
| 14.5 | 56 |
| 14.7 | 40 |
| 15.1 | 43 |
| 15.5 | 64 |
| 16.6 | 18 |
| 17.2 | 13 |
| 17.6 | 53 |
| 17.9 | 46 |
| 18.7 | 28 |
| 19.6 | 29 |
| 20.4 | 34 |
| 21.5 | 25 |
| 21.8 | 41 |
| 22.2 | 40 |
| 22.6 | 4 |
| 23.5 | 5 |
| 23.9 | 3 |
| 25.8 | 24 |
| 26.0 | 30 |
| 26.3 | 17 |
| 27.2 | 2 |
| 27.8 | 9 |
| 28.4 | 7 |
| 28.8 | 5 |
| 30.4 | 4 |
| 31.5 | 10 |
| 32.0 | 2 |
| 32.3 | 5 |
| 33.4 | 9 |
| 34.9 | 2 |
| 35.3 | 4 |
| 37.5 | 6 |
| 38.2 | 2 |
| 39.6 | 4 |

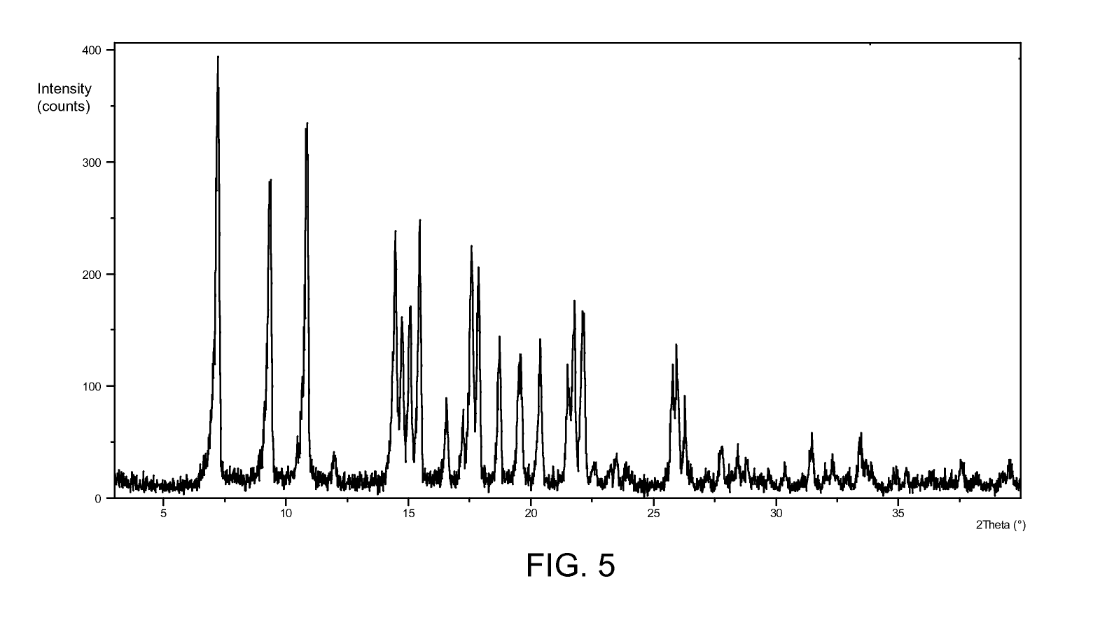

This salt Form A may be further characterized by an X-ray diffractogram as in FIG. 5.

The salt Form A may also be further characterized by the following ¹H NMR spectrum (DMSO-d₆, 400 MHz): δ=5.10 (dd, J=1.6 Hz, J=10.6 Hz, 1H); 4.38 (s br, 1 H); 3.96-3.85 (m, 1H); 3.83 (s, 2H); 3.52 (s, 2H); 2.20 (dd, J=1.6 Hz, J=13.7 Hz, 1H); 2.10 (s, 2H); 1.92-1.79 (m, 1H); 1.76-1.60 (m, 3H); 1.58-1.24 (m, 9H); 1.02-0.92 (m, 1H); 0.76-0.67 (m, 1H).

The salt Form A generally has an amount of water between 0.1 and 3% w/w, more preferably between 1 and 1.5% w/w measured by TGA.

The salt of saxagliptin with glycolic acid (1:1) hydrate Form A is advantageous since it is very soluble in water, and shows a high crystalline and chemical stability. As it is illustrated in the Examples it shows a better stability than saxagliptin free base H-1 and saxagliptin hydrochloride dihydrate H2-1 known in the art.

The salt of saxagliptin with glycolic acid (1:1) hydrate Form A may be prepared by a process comprising (a) crystallizing the salt from a solution of saxagliptin in isopropanol; and (b) isolating the salt obtained in step (a).

In a particular embodiment, the process further comprises the previous step of forming the salt by reacting saxagliptin monohydrate Form H-1 and glycolic acid in isopropanol.

In a particular embodiment, the isolation step (b) comprises cooling the mixture at room temperature and separating the product from the mixture by filtration or centrifugation. The salt thus obtained can be dried, preferably at a temperature comprise between room temperature and 40° C., preferably at 30° C., and under vacuum. Generally, the vacuum is comprised of 2 to 5 mbar.

Another aspect of the present invention is the provision of a cocrystal of saxagliptin hydrochloride and a compound selected from the group consisting of saxagliptin, glycolic acid, malonic acid, and urea; or a solvate thereof, or a hydrate thereof, for use as a medicament.

Another aspect of the present invention is the provision of a cocrystal of saxagliptin hydrochloride and a compound selected from the group consisting of saxagliptin, glycolic acid, malonic acid, and urea; or a solvate thereof, or a hydrate thereof, for use in the prevention and/or treatment of type 2 diabetes mellitus. This aspect can also be formulated as the use of a cocrystal of saxagliptin hydrochloride and a compound selected from the group consisting of saxagliptin, glycolic acid, malonic acid, and urea; or a solvate thereof, or a hydrate thereof, for the preparation of a medicament for the prophylactic and/or therapeutic treatment of type 2 diabetes in a mammal, including a human. The invention also relates to a method of treatment and/or prophylaxis of a mammal, including a human, suffering from or being susceptible to type 2 diabetes, said method comprises the administration to said patient of a therapeutically effective amount of the a cocrystal of saxagliptin hydrochloride and a compound selected from the group consisting of saxagliptin, glycolic acid, malonic acid, and urea; or a solvate thereof, or a hydrate thereof, together with pharmaceutically acceptable excipients or carders.

It is considered part of the invention, a salt of saxagliptin with glycolic acid (1:1) hydrate Form A, for use as a medicament.

It is considered part of the invention, a salt of saxagliptin with glycolic acid (1:1) hydrate Form A, for use in the prevention and/or treatment of type 2 diabetes. This aspect can also be formulated as the use of a salt of saxagliptin with glycolic acid (1:1) hydrate Form A, for the preparation of a medicament for the prophylactic and/or therapeutic treatment of type 2 diabetes in a mammal, including a human. The invention also relates to a method of treatment and/or prophylaxis of a mammal, including a human, suffering from or being susceptible to type 2 diabetes, said method comprises the administration to said patient of a therapeutically effective amount of a salt of saxagliptin with glycolic acid (1:1) hydrate Form A, together with pharmaceutically acceptable excipients or carriers.

Any of the cocrystals of saxagliptin of the invention disclosed above may be employed in various pharmaceutical formulations. Accordingly, a pharmaceutical composition comprising a cocrystal of saxagliptin hydrochloride and a compound selected from the group consisting of saxagliptin, glycolic acid, malonic acid and urea; or a solvate thereof, or a hydrate thereof, together with appropriate amounts of pharmaceutical excipients or carriers is also part of the invention.

Finally, it is also part of the invention a pharmaceutical composition comprising a salt of saxagliptin with glycolic acid (1:1) hydrate Form A, together with appropriate amounts of pharmaceutical excipients or carriers.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

### Example 1

### Preparation of saxagliptin HCl saxagliptin cocrystal (1:1) Form I

To a 2 mL eppendorf containing saxagliptin monohydrate, SAX(H-1), (25 mg, 0.07 mmol), saxagliptin hydrochloride dihydrate, SAXc(H2-1), (26 mg, 0.07 mmol) and three 4 mm stainless steel grinding balls, one drop of IPA was added. The reactor was stirred 45 minutes at a rate of 30 Hz (3×15 minutes). The product was dried under vacuum at room temperature.

Saxagliptin.saxagliptin hydrochloride (1:1) cocrystal Form I, was obtained as a white powder in a quantitative yield. It has been characterized by XRPD ¹H NMR (DMSO-d₆) and ¹H NMR (MeOD-d₄) and the compound obtained corresponds to the saxagliptin HCl.saxagliptin cocrystal (1:1) Form I.

Solubility and Stability in Water: 20 vol. in water at room temperature (20-25° C.): soluble (reprecipitation was not observed after 24 h).

Crystalline and chemical stability at 60° C.-37% RH: saxagliptin crystalline form I and the known saxagliptin crystalline forms H-1, H2-1 were stored at 60° C. with 37% relative humidity (RH). The samples were analyzed by XRPD and HPLC-MS to check crystalline transformation and chemical degradation. No crystalline transformations were observed for any of these forms during this study. However, chemical degradations were observed (see Table 6 below).

TABLE 6

| | Δ variation amidine Impurity % (HPLC-MS) | | |
|---|---|---|---|
| | 60° C.-37% RH | | |
| Crystalline form | 16 h | 168 h | 184 h |
| Comparative H-1 | 0.64% (A) | 2.68% (A) + 2.21% (B) | 2.86% (A) + |
| | | | 2.33% (B) |
| Comparative H2-1 | 0.14% (A) | 2.88% (A) + 0.5% (B) | 3.47% (A) + |
| | | | 0.61% (B) |
| Form I | 0.08% (A) | 0.38% (A) | 0.40% (A) |

(A) and (B) correspond to the following impurities:

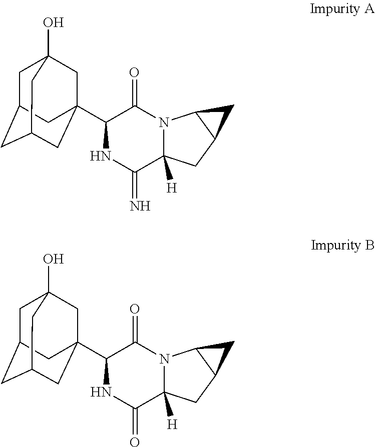 [SMILES: N=C1N[C@@H](C23CC4CC(CC(O)(C4)C2)C3)C(=O)N2[C@@H]1C[C@@H]1C[C@@H]12 ; O=C1N[C@@H](C23CC4CC(CC(O)(C4)C2)C3)C(=O)N2[C@@H]1C[C@@H]1C[C@@H]12]

From the previous results, it is clear that the degradation rate is much lower for cocrystal Form I than for the free base H-1 and hydrochloride H2-1. After 1 week of storage in these conditions, the color of the solid forms H-1 and H2-1 became yellowish, where as Form I remained white.

The stoichiometry of the cocrystal of saxagliptin HCl.saxagliptin cocrystal (1:1) Form I was confirmed by titration of a cocrystal of saxagliptin HCl and saxagliptin (1:1) Form I obtained by crystallization. Form I (100 mg, 0.15 mmol) was dissolved in water (10 mL) at room temperature. The resultant solution was titrated with a 0.1M NaOH aq. solution to determine the amount of HCl in Form I. The amount of 0.1 M NaOH aq. solution used in the analysis was 1.5 ml (0.15 mmol) which corresponds with a saxagliptin HCl.saxagliptin with 1:1 stoichiometry.

### Example 2

### Preparation of saxagliptin HCl.saxagliptin cocrystal (1:1) Form I

It was repeated Example 1 but using AcOEt saturated with water instead of IPA, yielding to the cocrystal of the title according to the characterization by XRPD.

### Example 3

### Preparation of saxagliptin HCl.saxagliptin cocrystal (1:1) Form I

To a round-bottom flask equipped with magnetic stirrer containing a solution of Saxagliptin monohydrate (200 mg, 0.60 mmol) in isopropanol (2.5 mL), was added dropwise a HCl solution (0.8 mL, 0.3 mmol) in water/isopropanol (30/50, vol/vol) (addition time 30 min). The solution was seeded, at the beginning of the addition and when a half of the HCl solution was added, with Form I. A white precipitate was formed and the slurry was stirred 2 hours at room temperature and 1 hour at 0-5° C. The solid was filtered with a sintered funnel (porosity 4), and dried under vacuum overnight at room temperature. Form I Saxagliptin—Saxagliptin.HCl (1:1) hydrate was obtained as a white powder (138 mg, 67% yield).

### Example 4

### Preparation of saxagliptin HCl.saxagliptin cocrystal (1:1) Form I

To a round-bottom flask equipped with magnetic stirrer containing a solution of Saxagliptin monohydrate (1 g, 3.00 mmol) in isopropanol (12.5 mL), was added dropwise a HCl solution (4 mL, 1.50 mmol) in water/isopropanol (30/50, vol/vol) (addition time 30 min). The solution was seeded, at the beginning of the addition and when a half of the HCl solution was added, with Form I. A white precipitate was formed and the slurry was stirred at room temperature overnight. The solid was filtered with a sintered funnel (porosity 3), and dried under vacuum overnight at room temperature. Form I Saxagliptin—Saxagliptin.HCl (1:1) hydrate was obtained as a white powder (572 mg, 56% yield).

### Example 5

### Preparation of saxagliptin HCl.glycolic acid cocrystal (1:1) hydrate Form II

To an assay tube equipped with magnetic stirrer containing saxagliptin hydrochloride dihydrate (20 mg, 0.05 mmol) and glycolic acid (5 mg, 0.07 mmol) was added xylene (1 mL, mixture of isomers). The resulting suspension was sonicated for 5 minutes and then stirred at room temperature overnight (20-25° C.). The solid was centrifuged at 14000 rpm for 15 min and dried under vacuum at room temperature. Saxagliptin hydrochloride.glycolic acid (1:1) cocrystal (Form II) was obtained as a white powder (19 mg, 83%) yield). It has been characterized by XRPD, ¹H NMR (DMSO-d₆) and ¹H NMR (MeOD-d₄) and the compound obtained corresponds to the saxagliptin HCl.glycolic acid cocrystal (1:1) hydrate Form II.

TGA analysis indicated that the loss of water started higher than room temperature (60° C.). So the amount of water is well defined and corresponds to a monohydrate (less water than saxagliptin HCl dihydrate H2-1).

Solubility: Soluble in 5 volumes of water at room temperature (same solubility than saxagliptin HCl dihydrate (H2-1)).

### Example 6

### Preparation of saxagliptin HCl.malonic acid cocrystal (3:2) hydrate Form III

To a 2 mL eppendorf containing saxagliptin hydrochloride dihydrate (20 mg, 0.05 mmol), malonic acid (4 mg, 0.04 mmol) and three 4 mm stainless steel grinding balls, one drop of MeOH was added. The reactor was stirred 45 minutes at a rate of 30 Hz (3×15 minutes) and at room temperature (without cooling or heating). The product was dried under vacuum at room temperature. Saxagliptin hydrochloride.malonic acid (1.5:1) Form III was obtained as a white powder in a quantitative yield. The compound obtained corresponds to the saxagliptin HCl.malonic acid (3:2) cocrystal hydrate Form III.

Solubility in water at room temperature: approximately 4 volumes of water (solubility similar to the form H2-1).

### Example 7

### Preparation of saxagliptin HCl.malonic acid cocrystal (3:2) hydrate Form III

To an assay tube equipped with magnetic stirrer containing saxagliptin monohydrate (20 mg, 0.06 mmol) and malonic acid (6 mg, 0.06 mmol) was added toluene (1 mL). The resultant suspension was stirred for a few minutes at 80° C. and seeded with cocrystal Form III obtained in the example 5. Then, hydrochloric acid 1.25M in IPA (58 μL, 0.07 mmol) was added. The resulting suspension was cooled to room temperature and stirred for 3 hours. The solid was filtered with a sintered funnel (porosity 4), washed with toluene (0.3 mL) and dried under vacuum at room temperature. Saxagliptin hydrochloride.malonic acid hydrate (3:2) Form III was obtained as a white powder (24 mg, 92% yield). It has been characterized by XRPD ¹H NMR (DMSO-d₆) and ¹H NMR (MeOD-d₄).

### Example 8

### Preparation of saxagliptin HCl.urea cocrystal (1:3) Form IV

To an assay tube equipped with magnetic stirrer containing saxagliptin hydrochloride dihydrate (20 mg, 0.05 mmol) and urea (9 mg, 0.15 mmol) was added AcOEt (1 mL). The resulting suspension was stirred at 65° C. overnight. The solid was filtered with a sintered funnel (porosity 4) and dried under vacuum at room temperature. Form IV saxagliptin hydrochloride.urea (1:3) was obtained as a white powder (25 mg, 93% yield). It has been characterized by XRPD, ¹H NMR (DMSO-d₆) and ¹H NMR (MeOD-d₄) and the compound obtained corresponds to saxagliptin HCl.urea cocrystal (1:3) Form IV.

The amount of water of Form IV is very low. According to the batch and the drying process, the amount of water was from 0.1 to 0.3 water equivalent (less water than H2-1: 2 eq. of water).

Solubility. Solubility in water at room temperature: Soluble in approximately 2.5 volumes of water (more soluble than H2-1).

### Example 9

### Preparation of a salt of saxagliptin with glycolic acid (1:1) hydrate Form A

To a round-bottom flask equipped with magnetic stirrer containing saxagliptin monohydrate (200 mg, 0.60 mmol) and glycolic acid (46 mg, 0.60 mmol) was added IPA (10 mL). The resultant suspension was stirred at 80° C. until complete dissolution and then cooled down to room temperature. A white precipitate was formed and the slurry was stirred at room temperature overnight. The solid was filtered with a sintered funnel (porosity 4), washed with IPA (1 mL) and dried under vacuum at 30° C. overnight. Form A saxagliptin.glycolic acid (1:1) was obtained as a white powder (157 mg, 67% yield). It has been characterized by XRPD, ¹H NMR (DMSO-d₆) and ¹H NMR (MeOD-d₄) and the compound obtained corresponds to the compound of the title.

Solubility: Soluble in 1 volume of water at room temperature (more soluble than H2-1).

Crystalline and chemical stability at 60° C.—37% RH: Saxagliptin crystalline forms H-1, H2-1 and A were stored at 60° C. with 37% RH. The samples were analyzed by XRPD and HPLC-MS to check crystalline transformation and chemical degradation. The results are shown in Table 7.

TABLE 7

| | |
|---|---|
| | Δ variation amidine |
| | Impurities A and B % (HPLC-MS) |
| Crystalline | 60° C.-37% RH |

| form | 16 h | 168 h |
|---|---|---|
| H-1 | 0.6% (A) | 2.7% (A) + 2.2% (B) |
| H2-1 | 0.1% (A) | 2.9% (A) + 0.5% (B) |
| Form A | 0.1% (A) | 0.7% (A) + 0.2% (B) |

### REFERENCES CITED IN THE APPLICATION

WO2001068603
WO2008131149
WO20100115974
WO2012017028
WO20111140328
WO2012047871
G. S. Jones et al., J. Org. Chem. 2011, vol. 76, pp. 10332-10337.

1. A cocrystal of saxagliptin hydrochloride and a compound selected from the group consisting of saxagliptin, glycolic acid, malonic acid and urea; or a solvate thereof, or a hydrate thereof.

2. The cocrystal according to claim 1, which is substantially free of any other crystalline form of an organic or inorganic compound.

3. The cocrystal according to claim 1, which is saxagliptin HCl.saxagliptin cocrystal 1:1.

4. The cocrystal according to claim 3, which is a hydrate.

5. The cocrystal according to claim 4, which is characterized by having an X-ray diffractogram that comprises characteristic peaks at approximately 6.8, 13.6, 14.3, 15.1 and 19.1 degrees 2 theta at a Cu—K_{α} radiation, λ=1.5406 Å.

6. The cocrystal according to claim 5, which is characterized by having an X-ray diffractogram that comprises characteristic peaks at approximately 6.8, 8.0, 8.6, 11.1, 13.6, 14.3, 14.5, 15.1, 15.7, 16.9, 17.2, 17.5, 17.6, 18.2, 18.5, 19.1, 20.6, 20.9, 21.4, 21.8, 22.6, 24.3, 25.0, 25.6, 26.0, 27.2, 27.5, 27.8, 29.1, 29.8, 30.6, 31.3, 32.2, 33.3, 34.5, 34.8, 35.7, and 37.2 degrees 2 theta (Cu—K_{α} radiation, λ=1.5406 Å) and without having any significant peak of other crystalline form of saxagliptin or a salt thereof or of an inorganic salt.

7. The cocrystal according to claim 1, which is saxagliptin HCl.glycolic acid cocrystal in a ratio of saxagliptin HCl.glycolic acid 1:1.

8. The cocrystal according to claim 7, which is saxagliptin HCl.glycolic acid cocrystal 1:1 hydrate characterized by having an X-ray diffractogram that comprises characteristic peaks at approximately 13.0, 14.4, 18.3, 18.9, 22.3 and 25.8 degrees 2 theta at a Cu—K_{α} radiation, λ=1.5406 Å.

9. The cocrystal according to claim 1, which is saxagliptin HCl.malonic acid cocrystal in a ratio saxagliptin HCl.malonic acid comprised of from 1:1 to 2:1.

10. The cocrystal according to claim 9, which is saxagliptin HCl.malonic acid cocrystal 3:2 hydrate and is characterized by having an X-ray diffractogram that comprises characteristic peaks at approximately 6.3, 14.4, 18.2, 22.4 and 25.8 degrees 2 theta at a Cu—K_{α} radiation, λ=1.5406 Å.

11. The cocrystal according to claim 1, which is saxagliptin HCl.urea cocrystal (1:3).

12. The cocrystal according to claim 11, which is characterized by having an X-ray diffractogram that comprises characteristic peaks at approximately 4.8, 9.7, 13.1, 17.8 and 20.4 degrees 2 theta at a Cu—K_{α} radiation, λ=1.5406 Å.

13. A salt of saxagliptin with glycolic acid 1:1 hydrate, characterized by having an X-ray diffractogram that comprises characteristic peaks at approximately 7.2, 10.9, 17.6, 17.9 and 21.8 degrees 2 theta at a Cu—K_{α} radiation, λ=1.5406 Å.

14. (canceled)

15. A method for the treatment and/or prevention of type 2 diabetes, comprising administering to a mammal, including a human, a therapeutically effective amount of a cocrystal of saxagliptin hydrochloride and a compound selected from the group consisting of saxagliptin, glycolic acid, malonic acid, and urea; or a solvate thereof, or a hydrate thereof as defined in claim 1, together with pharmaceutically acceptable excipients or carriers.

16. A method for the treatment and/or prevention of type 2 diabetes, comprising administering to a mammal, including a human, a therapeutically effective amount of a salt of saxagliptin with glycolic acid (1:1) hydrate as defined in claim 13, together with pharmaceutically acceptable excipients or carriers.

17. A pharmaceutical composition comprising a cocrystal of saxagliptin hydrochloride and a compound selected from the group consisting of saxagliptin, glycolic acid, malonic acid, and urea; or a solvate thereof, or a hydrate thereof, or a salt of saxagliptin with glycolic acid (1:1) hydrate characterized by having an X-ray diffractogram that comprises characteristic peaks at approximately 7.2, 10.9, 17.6, 17.9 and 21.8 degrees 2 theta at a Cu—K_{α} radiation, λ=1.5406 Å, together with appropriate amounts of pharmaceutical excipients or carriers.

18. A process for the preparation of the saxagliptin HCl.saxagliptin cocrystal as defined in claim 3, which comprises providing a solution of saxagliptin in a solvent system selected from the group consisting of water/(C₁-C₆)-alcohol, (C₁-C₆)-alcohol, methylisobutylketone, and ethyl acetate; adding an solution of hydrochloric acid in water or water/(C₁-C₆)-alcohol; and subsequently isolating the precipitated compound thus obtained.

19. The process according to claim 18, wherein the (C₁-C₆)-alcohol is isopropanol.

20. The cocrystal according to claim 2, which is saxagliptin HCl.saxagliptin cocrystal 1:1.
